(19) United States
(12) Patent Application Publication
(10) Pub. No.: US 2024/0390135 A1
(43) Pub. Date: Nov. 28, 2024

(54) Single Frame Tethered Transcatheter Heart Valve

(71) Applicant: Tendyne Holdings, Inc., St. Paul, MN (US)

(72) Inventors: William H. Peckels, Robbinsdale, MN (US); Heath Marnach, Minneapolis, MN (US); Taylor Nasby, Savage, MN (US); Amy Marie Danielson, St. Paul, MN (US)

(73) Assignee: Tendyne Holdings, Inc., St. Paul, MN (US)

(21) Appl. No.: 18/632,386

(22) Filed: Apr. 11, 2024

Related U.S. Application Data
(60) Provisional application No. 63/504,039, filed on May 24, 2023.

(51) Int. Cl. A61F 2/24

(52) U.S. Cl. CPC A61F 2/2418; A61F 2220/0008; A61F 2220/0075; A61F 2250/006

(57) ABSTRACT

According to one aspect of the disclosure, a prosthetic atrioventricular valve system includes a prosthetic atrioventricular valve having a self-expandable frame with a collapsed and expanded condition. In the expanded condition, the frame includes (i) a body portion sized to be positioned within, and in contact with, a native atrioventricular valve annulus, (ii) an atrial flare portion positioned in an inflow direction relative to the body portion, the atrial flare portion having an outer diameter that is greater than an outer diameter for the body portion, (iii) a plurality of commissure attachment features (“CAFs”) extending in an outflow direction from the body portion, and (iv) a commissure support ring coupled to and circumferentially supporting the plurality of CAFs. A plurality of prosthetic leaflets may be mounted to the CAFs. The body portion, the atrial flare portion, and the plurality of CAFs may be formed integrally.

### CROSS-REFERENCE TO RELATED APPLICATIONS

This application claims the benefit of the priority date of U.S. Provisional Patent Application No. 63/504,039, filed May 24, 2023, the disclosure of which is hereby incorporated by reference herein.

### BACKGROUND OF THE DISCLOSURE

The present disclosure relates to expandable prosthetic heart valves, and more particularly, to apparatus and methods related to implanting an expandable prosthetic heart valve within a native annulus of a patient.

Prosthetic heart valves that are collapsible to a relatively small circumferential size can be delivered into a patient less invasively than valves that are not collapsible. For example, a collapsible and expandable valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible and expandable prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed to reduce its circumferential size.

When a collapsed prosthetic valve has reached the desired implant site in the patient (e.g., at or near the native annulus of the patient's heart valve that is to be repaired by the prosthetic valve), the prosthetic valve can be deployed or released from the delivery apparatus and expanded to its full operating size. For balloon-expandable valves, this generally involves releasing the entire valve, assuring its proper location, and then expanding a balloon positioned within the valve stent. For self-expanding valves, on the other hand, the stent automatically expands as the stent is released from the delivery apparatus.

The clinical success of collapsible and expandable heart valves is dependent, in part, on the anchoring of the valve within the native valve annulus. Self-expanding valves typically rely on the radial force exerted by expanding the stent against the native valve annulus to anchor the prosthetic heart valve. However, if the radial force is too high, the heart tissue may be damaged. If, instead, the radial force is too low, the heart valve may move from its deployed position and/or migrate from the native valve annulus, for example, into the left atrium.

Movement of the prosthetic heart valve or its sub-optimal placement within the anatomy may result in the leakage of blood between the prosthetic heart valve and the native valve annulus. This phenomenon is commonly referred to as paravalvular leakage (PVL). In mitral valves, paravalvular leakage enables blood to flow from the left ventricle back into the left atrium during ventricular systole, resulting in reduced cardiac efficiency and strain on the heart muscle.

Anchoring prosthetic heart valves within the native valve annulus of a patient, especially within the native mitral valve annulus, can be difficult. The native mitral valve annulus, for instance, has reduced calcification or plaque compared to the native aortic valve annulus which can make for a less stable surface to anchor the prosthetic heart valve. For this reason, collapsible and expandable prosthetic mitral valves often include additional anchoring features such as barbs that engage underneath the annulus and/or coils that capture native leaflets, or that wrap around chordae tendineae, thereby stabilizing the prosthetic heart valve within the native annulus.

Furthermore, prosthetic mitral and prosthetic tricuspid valves are typically much larger than prosthetic aortic and prosthetic pulmonary valves, at least in part due to the corresponding larger sizes of the native valves. To achieve both the strong anchoring typically required within native atrioventricular valves, as well as good hemodynamic performance of the prosthetic leaflets, prosthetic atrioventricular valves often include an outer frame that is primarily used for anchoring, and an inner frame that is primarily used for receiving the prosthetic heart valve leaflets. While this configuration has achieved success among the general population, there is a patient population that includes atrioventricular valves that are smaller than average, including because of (i) annular calcification or (ii) smaller anatomic sizes. These patients may not be candidates to receive a transcatheter, dual-framed prosthetic atrioventricular valve because even the smallest dual-frame prosthetic heart valve commercially available may be too large for this patient population. Such patients may be treated via a more traditional surgical valve replacement, but not all patients are candidates for surgical valve replacement. In fact, the type of patient most in need of a prosthetic valve may be likely to be too frail to survive the open heart surgery required for a surgical valve replacement. As a result, there are patients who would benefit from a prosthetic heart valve, but who are not candidates for surgical valve replacement and for whom commercially available transcatheter heart valves are too large for proper functioning. Thus, it would be desirable to have a device suited to effectively treat this patient population.

### SUMMARY OF THE DISCLOSURE

According to one aspect of the disclosure, a prosthetic atrioventricular valve system includes a prosthetic atrioventricular valve having a self-expandable frame with a collapsed and expanded condition. In the expanded condition, the frame includes (i) a body portion sized to be positioned within, and in contact with, a native atrioventricular valve annulus, (ii) an atrial flare portion positioned in an inflow direction relative to the body portion, the atrial flare portion having an outer diameter that is greater than an outer diameter for the body portion, (iii) a plurality of commissure attachment features (“CAFs”) extending in an outflow direction from the body portion, and (iv) a commissure support ring coupled to and circumferentially supporting the plurality of CAFs. A plurality of prosthetic leaflets may be mounted to the CAFs. The body portion, the atrial flare portion, and the plurality of CAFs may be integral with each other.

The commissure support ring may be integral with the body portion. The commissure support ring may be formed by at least one row of diamond-shaped cells. The cells of the commissure support ring may be positioned between adjacent pairs of the plurality of CAFs. The at least one row of diamond-shaped cells forming the commissure support ring may be the only row of diamond-shaped cells forming the commissure support ring. Each cell of the commissure support ring may have an inflow apex, and no inflow apex of any cell of the commissure support ring may be directly coupled to the body portion. Selected struts may directly couple the commissure support ring to the body portion, the selected struts attaching to the commissure support ring at locations between selected circumferentially adjacent pairs of cells of the commissure support ring. The frame may be a first frame, and the prosthetic atrioventricular valve may not include a second frame. The commissure support ring may not be integral with the frame. The commissure support ring may be assembled to the frame so that the commissure support ring is aligned with, and radially outside of, the plurality of CAFs.

The frame may include a plurality of tether struts, and the system may include a tether secured the plurality of tether struts at a terminal end of the plurality of tether struts. The plurality of tether struts may be integral with the frame, each of the plurality of tether struts extending in the outflow direction from a corresponding one the plurality of commissure attachment features. The plurality of tether struts may include exactly three tether struts and the plurality of CAFS may include exactly three commissure attachment features. The plurality of tether struts may be integral with the commissure support ring, each of the plurality of tether struts extending in the outflow direction form the commissure support ring. Each of the tether struts may terminate at a free end, each of the free ends including at least one aperture for receiving a connecting suture therethrough. The system may include an epicardial anchor configured to attach to the tether. The system may further include a skirt coupled to an exterior surface of the atrial flare portion. The atrial flare portion may be rotationally symmetric. The system may further include a plurality of cups coupled to the body portion, each of the plurality of cups being coupled to a cell of the bod portion and including two patch portions, the two patch portions begin coupled to each outer at an inflow portion of the two patch portions, but uncoupled to each other at an outflow end portion, so that retrograde blood may flow into each cup via the uncoupled outflow end portion to force the two patch portions to billow away from each other. The two patch portions of each of the plurality of cups may be generally triangular.

### BRIEF DESCRIPTION OF DRAWINGS

FIG. 1 is a highly schematic cutaway view of the human heart, showing two approaches for delivering a prosthetic mitral valve to an implantation location.

FIG. 2 is a highly schematic representation of a native mitral valve and associated cardiac structures.

FIG. 3 is an exploded view of a double-framed, tether-anchored, prosthetic atrioventricular valve according to the prior art.

FIGS. 4-5 are side and top (inflow) views, respectively, of a prosthetic heart valve according to an aspect of the disclosure.

FIG. 6 illustrates a cut pattern of the frame of the prosthetic heart valve of FIGS. 4-5.

FIG. 7 is a schematic illustration of a sealing feature provided on the frame of the prosthetic heart valve of FIGS. 4-5, shown during ventricular diastole.

FIG. 8 is a schematic illustration of the sealing feature of FIG. 7, shown during ventricular systole.

FIG. 9 is a highly schematic view of the prosthetic heart valve of FIGS. 4-5 implanted within a native annulus to show possible relative positioning between the sealing feature of FIGS. 7-8 and the native anatomy.

FIG. 10 is a perspective view of a frame of a prosthetic heart valve, according to another aspect of the disclosure, in an expanded condition.

FIG. 11 is a perspective view of a prosthetic heart valve that incorporates the frame of FIG. 10.

FIG. 12 is a cut pattern for an alternate frame compared to that shown in FIG. 10.

FIG. 13 is a cut pattern for another alternate frame compared to that shown in FIGS. 10 and 12.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic atrioventricular valve, the inflow end is the end nearer the atrium while the outflow end is the end nearer the ventricle when the prosthetic heart valve is implanted as intended. The intended position and orientation are used for the convenience of describing the valve disclosed herein, however, it should be noted that the use of the valve is not limited to the intended position and orientation but may be deployed in any type of lumen or passageway. For example, although the prosthetic heart valve is generally described herein as a prosthetic mitral valve, the same or similar structures and features can be employed in other heart valves, such as the tricuspid (i.e., right atrioventricular) valve. As used herein, the terms “substantially,” “generally,” “approximately,” and “about” are intended to mean that slight deviations from absolute are included within the scope of the term so modified. As used herein, the stent frame may assume an “expanded state” and a “collapsed state,” which refer to the relative radial size of the frame/stent.

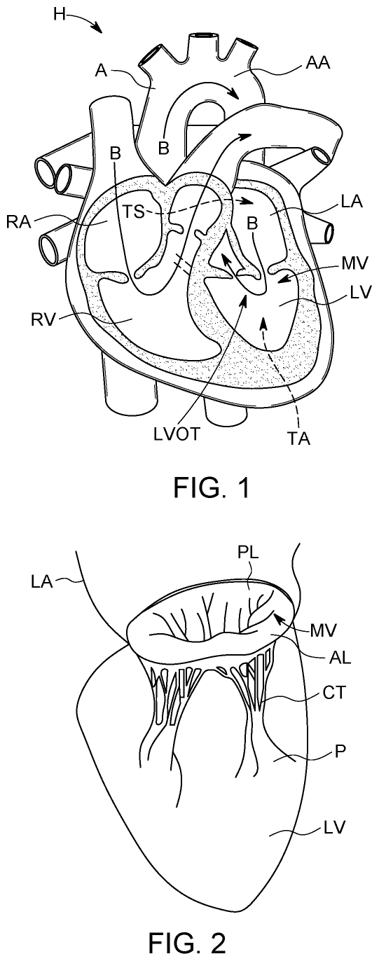

FIG. 1 is a schematic cutaway representation of a human heart H. The human heart includes two atria and two ventricles: right atrium RA and left atrium LA, and right ventricle RV and left ventricle LV. Heart H further includes aorta A, aortic arch AA and left ventricular outflow tract LVOT. Disposed between left atrium LA and left ventricle LV is mitral valve MV. The mitral valve, also known as the bicuspid valve or left atrioventricular valve, is a dual-flap that opens as a result of increased pressure in left atrium LA as it fills with blood. As atrial pressure increases above that in left ventricle LV, mitral valve MV opens and blood flows from the left atrium into the left ventricle. When left ventricle LV contracts during systole, blood is pushed from the left ventricle, through left ventricular outflow tract LVOT and into aorta A. Blood flows through heart H in the direction shown by arrows “B”.

A dashed arrow, labeled “TA”, indicates a transapical approach of implanting a prosthetic heart valve, in this case to replace the mitral valve. In the transapical approach, a small incision is made between the ribs of the patient and into the apex of left ventricle LV to deliver the prosthetic heart valve to the target site. A second dashed arrow, labeled “TS”, indicates a transseptal approach of implanting a prosthetic heart valve in which the delivery device is inserted into the femoral vein, passed through the iliac vein and the inferior vena cava into right atrium RA, and then through the atrial septum into left atrium LA for deployment of the valve. Other approaches for implanting a prosthetic heart valve are also possible and may be used to implant the collapsible prosthetic heart valve described in the present disclosure.

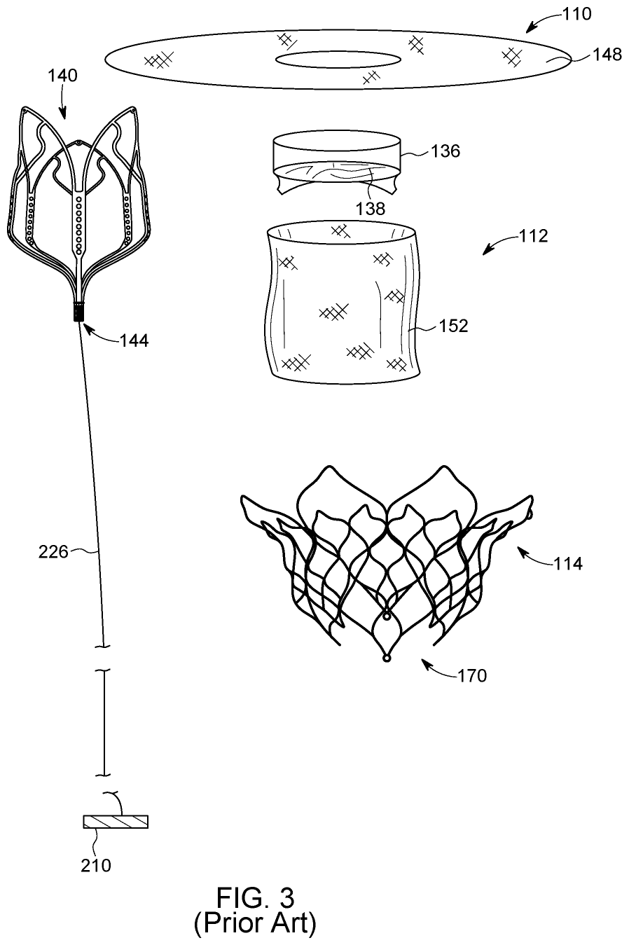

FIG. 2 is a more detailed schematic representation of native mitral valve MV and its associated structures. As previously noted, mitral valve MV includes two flaps or leaflets, posterior leaflet PL and anterior leaflet AL, disposed between left atrium LA and left ventricle LV. Cord-like tendons, known as chordae-tendineae CT, connect the two leaflets to the medial and lateral papillary muscles P. During atrial systole, blood flows from higher pressure in left atrium LA to lower pressure in left ventricle LV. When left ventricle LV contracts during ventricular systole, the increased blood pressure in the chamber pushes the posterior and anterior leaflets to close, preventing the backflow of blood into left atrium LA. Since the blood pressure in left atrium LA is much lower than that in left ventricle LV, the leaflets attempt to evert to low pressure regions. Chordae tendineae CT may help to prevent the eversion by becoming tense, thus pulling on the leaflets and holding them in the closed position.

FIG. 3 is an exploded view of a double-framed, tether-anchored, prosthetic heart valve 110 according to the prior art. Prosthetic heart valve 110 is a prosthetic mitral valve, but it should be understood that similar or identical configurations may be applied to a prosthetic tricuspid valve. Prosthetic heart valve 110 includes an inner structure or valve assembly 112 and an outer structure or assembly 114. Prosthetic heart valve 110 may be coupled to a tether 226 and a tether anchor 210.

Inner assembly 112 may include an inner stent or frame 140, outer wrap (e.g. tissue and/or fabric wrap) 152 which may be generally cylindrical, and prosthetic leaflet structure 136 (including articulating prosthetic leaflets 138 that define a valve function). Leaflet structure 136 may be coupled (e.g., via suturing) to inner frame 140, and may use parts of inner frame 140 for this purpose, although method of attachment other than sutures may be suitable. Inner assembly 112 is disposed and secured within outer assembly 114, as described in more detail below. The prosthetic leaflets 138 may be formed of any suitable material, such as synthetic fabrics and/or bioprosthetic tissue (e.g. porcine pericardial tissue).

Outer assembly 114 includes outer stent or frame 170. Outer frame 170 may also have in various embodiments outer and/or inner frame coverings of tissue and/or fabric (not pictured).

Tether 226 may be connected to prosthetic heart valve 110 by inner frame 140. Thus, inner frame 140 includes tether connecting or clamping portion 144 by which inner frame 140, and by extension prosthetic heart valve 110, is coupled to tether 226.

Generally, the inner frame 140 may be formed from a milled or laser-cut tube of a shape memory material such as, for example, nitinol. The inner frame 140 may be conceptually divided into four portions corresponding to functionally different portions of inner frame: an apex portion (the top of the inner frame in the view of FIG. 3), a body portion (the axial center of the inner frame in the view of FIG. 3, a strut portion (the inwardly tapering bottom portion in the view of FIG. 3), and a tether connecting portion (the bottom or terminal outflow end of the inner frame in the view of FIG. 3).

The inflow/apex portion of inner frame 140 may include struts with contouring (e.g. general “U”-shapes or serpentine shapes) to which bellies of three prosthetic leaflets 138 may be sutured. The body portion of inner frame 140 may include six longitudinal posts or bars with a column of apertures for receiving sutures to attach the prosthetic leaflets 138 and/or the outer frame 170 to the inner frame 140. The strut portion may function to connected the body portion to the tether connecting portion 144. The tether connecting portion 144 may include longitudinal extensions of the struts, connected circumferentially to one another by pairs of “V”-shaped connecting members, which may be referred to herein as “micro-V's.”

Connecting portion 144 is configured to be radially collapsed by application of a compressive force, which causes the micro-V's to become more deeply “V”-shaped, with each pair of vertices moving closer together longitudinally and the open ends of the “V” shapes moving closer together circumferentially. When collapsed, connecting portion 144 can clamp or grip one end of tether 226, either connecting directly onto a tether line (e.g., braided filament line) or onto an intermediate structure, such as a polymer or metal piece that is, in turn, firmly fixed to the tether line. The foregoing is merely exemplary and other techniques can be used to connect tether 226 to connecting portion 144.

The outer frame 170 can be formed from a milled or laser-cut tube of a shape memory material such as, for example, nitinol. Outer frame 170 may be conceptually divided into a coupling portion (toward the bottom of the view in FIG. 3), a body portion (the main axial central portion in FIG. 3), and a flared portion 173 (the top or terminal inflow end in FIG. 3). The bottom coupling portion may include multiple openings or apertures by which outer frame 170 can be coupled to inner frame 140, e.g., via suturing through the holes or apertures in the posts of the body portion of the inner frame 140. The flared portion of the outer frame 170 may be asymmetric to better match the typical anatomical topography of the mitral valve, including the portions of the left atrium just above the mitral valve annulus. The outer frame 170 may include one or more indicia to assist with achieving the desired rotational orientation of the outer frame 170 relative to the patient's anatomy during implantation.

It should be understood that, when the prosthetic heart valve 110 is assembled, the prosthetic leaflets 138 are positioned radially inside the inner frame 140, and the inner frame is positioned radially inside the outer frame 170, with the inner and outer frames coupled together on the ventricular side of the mitral valve annulus when implanted, so that a radial gap is maintained between the inner frame 140 and the outer frame 170 at the level of the mitral valve annulus when implanted. With this configuration, the outer frame 170 primarily functions to anchor the prosthetic heart valve 110 within the mitral valve annulus, and if the outer frame 110 is deformed by forces exerted during normal operation of the heart, the inner frame 140 (and thus the prosthetic leaflets 138 mounted therein) may avoid any significant deformation.

Anchor 210 may take various different configurations. For example, for a transapical procedure, the anchor 210 may be a rigid (e.g., plastic) disk that may be slid over the tether 226 until the anchor 210 contacts the outer surface of the heart and the transapical puncture. The anchor 210 may include fabric or other material to help seal (including via tissue ingrowth) the transapical puncture. After the anchor is 210 is in contact with the outside of the heart, the tether 226 may be tensioned and the anchor 210 may be locked to the tether 226 at the desired tension, with excess length of the tether being cut away. In other embodiments, for example in a transseptal delivery, the anchor 210 may be formed as an expandable (e.g. a nitinol frame or braided nitinol member) anchor that can be positioned on the outside of the heart by making an internal transapical puncture, without the need for any chest incision.

Examples of double-framed prosthetic mitral valves are described in more detail in U.S. Pat. No. 11,096,782. Examples of rigid epicardial anchors are described in more detail in U.S. Pat. No. 10,610,354. Examples of collapsible epicardial anchors are described in more detail in U.S. Patent Application Publication Nos. 2021/0369257 and 2022/0054259. Each of the four documents mentioned above is hereby incorporated by reference herein.

The dual-frame prosthetic mitral valve 110 described above has been found to work well in maintaining superior hemodynamics and valve function, but as noted above, the inclusion of two separate overlapping frames limits the patient population that can be treated while maintaining proper valve hemodynamics. As further noted above, patients that are not a candidate for surgical valve replacement that also have small mitral annuli often cannot be treated with the minimally invasive dual-frame design of prosthetic mitral valve 110. For example, the small annular geometries of these patients can limit the hemodynamic ability of the prosthetic heart valve 110 due to ovalization and size constraints. In other words, a small mitral valve annulus may overly compress the prosthetic mitral valve 110, and cause the prosthetic heart valve 110 to ovalize to a large enough extent that prevents proper leaflet coaptation, even though the prosthetic heart valve 110 is configured to withstand such forces in patients with larger mitral valve annuli. The prosthetic heart valve(s) described below include new designs, such as a single frame design, that may allow treatment of a greater patient population (particularly those with small mitral valve annuli) to reduce mitral regurgitation while maintaining proper hemodynamic performance of the prosthetic heart valve. In addition, the prosthetic heart valve(s) described below may help to promote valve-in-valve implantation and left atrial appendage (LAA) device implantation capabilities. Still further, the prosthetic heart valve(s) described below may help to reduce the cost of the prosthetic heart valve, including for example by having only a single frame may reduce manufacturing time/costs and material costs.

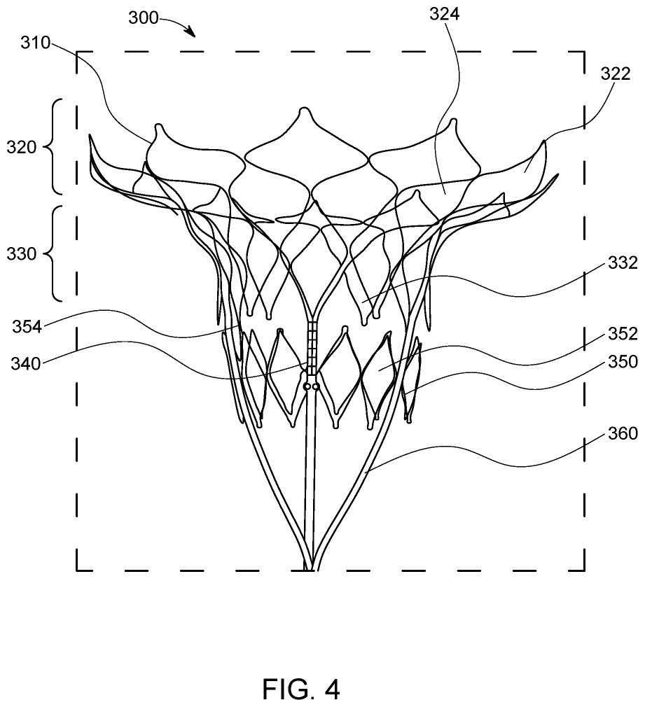

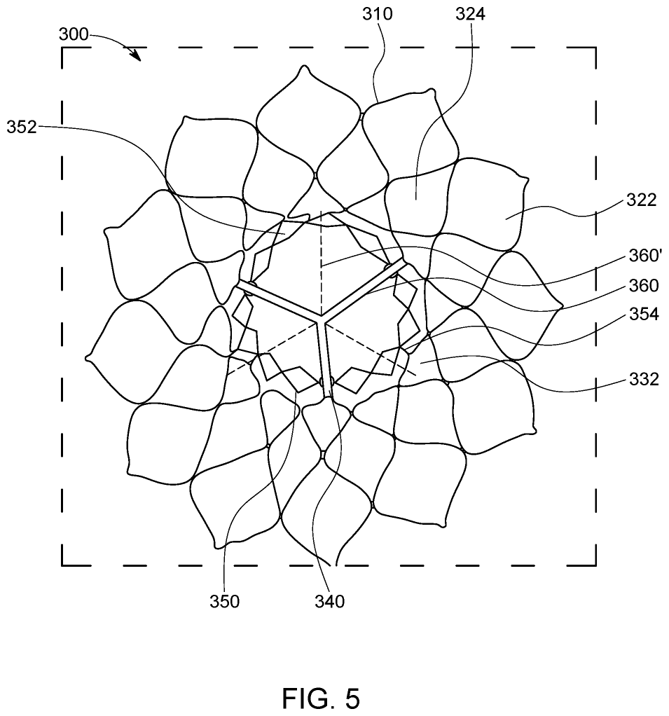

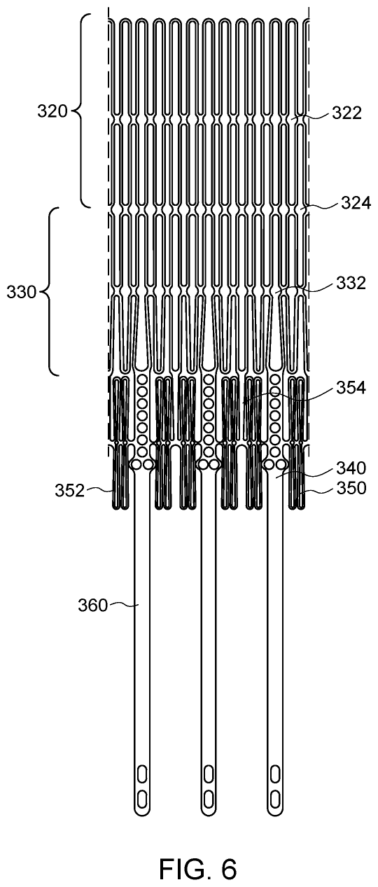

FIGS. 4-5 are side and top (inflow) views of a prosthetic mitral valve 300 according to an aspect of the disclosure. It should be understood that, for clarity of illustration, the prosthetic heart valve 300 shown in FIGS. 4-5 omit from the view the “soft” components of the prosthetic heart valve, including prosthetic leaflets and cuff materials. Compared to prosthetic heart valve 110, one difference of prosthetic heart valve 300 is that prosthetic heart valve 300 includes a single frame 310 that is configured to provide both anchoring within the native mitral valve annulus, as well as directly supporting the prosthetic leaflets within the single frame 310. FIG. 6 illustrates a cut pattern of the frame 310, which may generally correspond to the shape of the frame 310 as if collapsed, cut axially, and laid flat on a table. Preferably, frame 310 is formed of a self-expanding and/or super-clastic material, such as a nickel titanium alloy like nitinol. Frame 310 is shown in FIGS. 4-5 in an expanded condition (e.g. shape-set and/or deployed configuration in the absence of applied forces), and it should be understood that the frame 310 may be collapsed down to a smaller radial size for positioning within a catheter for transcatheter (e.g. transapical or transseptal) delivery to the mitral valve, and the frame 310 may be re-expanded upon deployment from the delivery catheter. In some embodiments, the frame 310 may be formed as an integral unit, for example via laser-cutting a tube of nitinol and then setting the desired expanded shape of the frame 310 via heat treatment. Although the term “single” frame is used herein to described prosthetic heart valve 300, as will become clear below, the frame may be formed as multiple pieces that are coupled together. Rather, the term “single” frame is intended to refer to the fact that the frame 310 does not include two separate frames with significant size that radially overlap one another along a significant portion of the axial length of each frame.

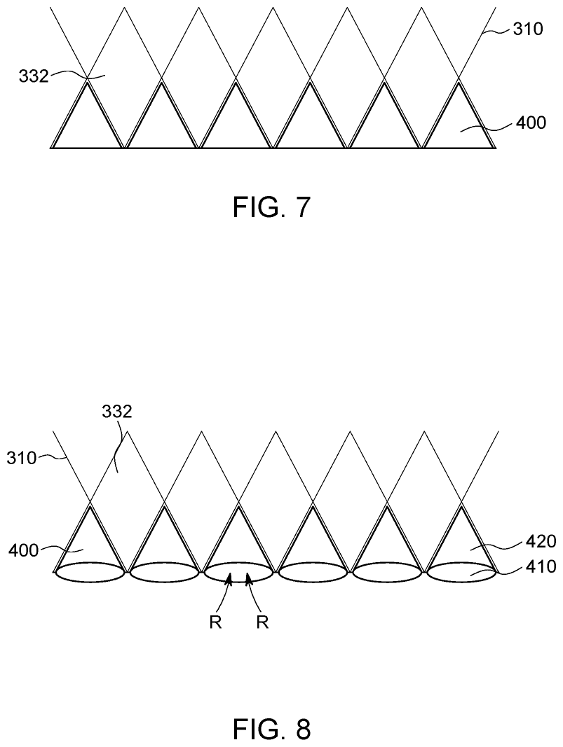

Referring to FIGS. 4-6, the inflow end of the frame 310 may include an atrial cuff or flare section 320. In the illustrated embodiment, the atrial flare 320 is formed partially or entirely by an inflow-most circumferential row of generally diamond-shaped cells 322. In the expanded condition, the atrial flare 320 may flare radially outwardly from the remainder of the frame 310. In the illustrated embodiment, the inflow tips of cells 322 may turn radially inwardly so that the terminal end of the atrial flare 320 presents a generally atraumatic contour to help avoid cutting, damaging, or otherwise irritating tissue that the atrial flare 320 contacts. The atrial flare 320 may be formed, in part, by a second circumferential row of generally diamond-shaped cells 324 that transition between the atrial flare section 320 and the main body section 330, described in more detail below. As best shown in FIG. 5, in the expanded condition of the frame 310, the atrial flare 320 is generally circular and/or symmetric about the central longitudinal axis of the frame 310. This is in contrast to the atrial flare of outer frame 170, which as described above is asymmetric, for example with an oval shape, a “D”-shape, or similar shape to generally match the contours typically found in native mitral valves. The inclusion of circular and/or rotationally symmetric atrial flare 320 may eliminate the need for rotational “clocking” of the prosthetic heart valve 300 during delivery and deployment. In other words, any rotational position (about the central longitudinal axis) of the prosthetic heart valve 300 should work just as well as any other rotational position, eliminating the need to position the prosthetic heart valve 300 in one particular rotational orientation (or a small range of rotational orientations) with respect to the native mitral valve. The elimination of this requirement may simplify the delivery procedure for medical personnel, which may also reduce procedural time which is typically beneficial for patients and medical personnel alike. Furthermore, compared to the atrial flare of outer frame 170, the atrial flare section 320 of prosthetic heart valve 300 may be shorter. In other words, when the prosthetic heart valve 300 is implanted, the terminal inflow end of the frame 310 may extend a distance “upwardly” within the left atrium a smaller distance than prosthetic heart valve 110. In particular, upon implantation of prosthetic heart valve 300, there may be no structure of prosthetic heart valve 300 that extends over the ostium to the LAA. By providing clearance to the LAA ostium, future procedures with the LAA, such as occlusion by delivering and implanting a LAA occluder into the LAA, will not be hindered by structure of the frame 310. Another benefit of this height differential is that, if frame 310 were to extend to partially block the LAA ostium, blood clotting at that structure may be more likely, which could lead to an embolism. Thus, the smaller height of atrial flare 320 may mitigate clot formation on the frame 310 near the LAA ostium. While the symmetric nature of the atrial flare 320 may provide certain benefits, it may conform to the native anatomy less precisely than the atrial flare of outer cuff 170, which could increase the likelihood of PV leak. However, as described in greater detail below in connection with FIGS. 7-9, PV leak mitigation features may be (but need not be) provided on prosthetic heart valve 300 to counteract a possible increased likelihood of PV leak.

Referring to FIGS. 4 and 6, the frame 310 may include a body portion 330 that couples to and/or transitions into the atrial flare section 320. In the illustrated embodiment, the body portion 330 is formed by a circumferential row of generally diamond-shaped cells 332. Transition cells 324 may also be considered to be part of the body portion 330. In the expanded condition of frame 310, the body portion 330 may be generally cylindrical, with an outer diameter that is significantly smaller than the outer diameter of the atrial flare portion 320. Functionally, the body section 330 may be configured to sit within the mitral valve annulus to help seal (along with other sealing features described below) the prosthetic heart valve 300 within the native valve annulus.

Still referring to FIGS. 4-6, the frame 310 may include a plurality of commissure attachment features (CAFs) 340 positioned within and/or adjacent to the main body section 330. The frame 310 may include the same number of CAFs 340 as prosthetic leaflets. In the illustrated example, the prosthetic heart valve is configured to include three prosthetic leaflets (which may be similar to, or the same as, prosthetic leaflets 138), and thus the frame 310 includes three CAFs. As best shown in FIG. 6, the CAF 340 are preferably positioned at equal intervals (e.g. every 120 degrees) around the circumference of the frame 310. In the illustrated embodiment, each CAF 340 take the form of a strut or post that extends in the outflow direction from an outflow apex of one of the cells 332 of the main body 330. Each CAF 340 may include a plurality of holes or apertures that may receive sutures to assist with coupling the commissures of the prosthetic leaflets to the frame 310. In the illustrated embodiment, the apertures are generally provided as a single column of apertures, although a pair of apertures is provided at the end of the column. This pair or apertures may provide additional ability to couple (e.g., via sutures) the tips of the leaflet commissures, for example by providing extra area to distribute stitches. However, it should be understood that the pair of apertures may be omitted so that only a single column of apertures is provided, or other specific number and positioning of apertures may otherwise be provided as desired. Although not shown, the frame 310 may include an interior cuff (e.g. tissue or fabric), and the bellies of the prosthetic leaflets may be coupled to the frame 310 via that interior cuff (e.g. via suturing) with or without direct connection of the prosthetic leaflet bellies to the frame 310. In the illustrated embodiment, the CAFs 340 are formed integrally with other portions of the frame 310, including the atrial flare 320 and the main body 330. With this configuration, the prosthetic leaflets (e.g. at commissures between pairs of adjacent prosthetic leaflets) are directly attached to the same frame 310 that is responsible for anchoring (e.g. direct contact with) the native valve annulus. With this configuration, the prosthetic heart valve 300 may have a significantly smaller profile (e.g. outer diameter) that allows for treatment of patients with relatively small sizes of mitral valve annuli, particularly compared to the dual-frame embodiment of prosthetic heart valve 110 that separates the anchoring and leaflet-mounting functionality into two separate, radially overlapping frames. In other words, this single frame design (which includes the below-described commissure support ring being formed integrally with, or separate from, the remainder of the frame 310) allows more patients to be treated since the prosthetic heart valve 300 is able to treat smaller annular geometries while maintaining proper hemodynamic performance. This is possible, at least in part, because the prosthetic heart valve 300 is less constrained (compared to prosthetic heart valve 110 and other similar dual-frame embodiments) by the native size of the annulus, and less likely to be impacted by ovalization from a mismatch between frame-annulus sizing.

Frame 310 may include two main additional components, including a commissure ring 350 and tether struts 360, which are described generally in connection with FIGS. 4-6.

Tether struts 360 may be generally axially-extending struts 360 that, in the expanded condition of the frame 310, transition radially inwardly to a terminal outflow end of the frame 310 where an end of the tether (e.g. tether 226) couples to the frame 310. In the illustrated example of FIGS. 4-6, each tether strut 360 is an outflow-extending continuation of the CAFs 340, with each tether strut 360 terminating in a free end that may include one or more apertures to receive sutures to help coupled the tether to the frame 310. The tether struts 360 include at least two notable differences compared to the tether struts of inner frame 140. First, frame 310 may include only three tether struts 360, compared to the six tether struts provided with inner frame 140. Second, while the tether struts of inner frame 140 are describe above as terminating in a cylindrical compressible connection portion formed of “V”-shaped struts, the tether struts 360 are, in this particular embodiment, simpler in the sense that they simply terminate in a free end with apertures. To connect the tether to the frame 310, the end of the tether may be placed interior to the three tether struts 360, and the tether may be sutured to the frame 310, including by passing sutures through the apertures in the tether struts 360.

By including fewer tether struts 360, there is less frame structure leading to the interior of the prosthetic heart valve 300 from the outflow end. If it becomes necessary to implant a second prosthetic heart valve after prosthetic heart valve 300 begins to fail, such a “valve-in-valve” procedure may be more easily performed because a catheter containing the secondary valve may be more easily navigated into the center of the prosthetic heart valve 300. In other words, after prosthetic heart valve 300 has been functioning for years or decades, it may begin to lose efficiency. In that situation, in a later procedure, a second collapsible and expandable prosthetic heart valve may be implanted within prosthetic heart valve 300, with access to that second-stage implantation being easier as there is less “blocking” structure leading to the interior of the prosthetic heart valve 300 compared to prosthetic heart valve 110.

Although FIGS. 4-6 illustrate the tether struts 360 being formed as continuations of the CAFs 340, other configurations may be suitable. For example, in other embodiments, the tether struts 360 may extend directly from portions of the commissure ring 350 (which is described in greater detail below) instead of directly from the CAFs 340. This alternate positioning of tether struts 360 is shown in FIG. 5 with dotted lines that represent the location of alternate tether struts 360′. It should be understood that, if the tether struts are provided at the alternate location 360′, the tether struts actually shown in FIGS. 4-6 would preferably be omitted, so that still only three tether struts are provided with the frame 310.

Still referring to FIGS. 4-6, the final main component of frame 310 is a commissure support ring 350, which may be referred to herein simply as a commissure ring or support ring. In the illustrated embodiment, the commissure ring 350 is formed integrally with the remainder of the frame 310, and takes the form of a single row of generally diamond-shaped cells 352. In the illustrated embodiment, there are four cells 352 positioned between, and connecting, each adjacent pair of CAFs 340. Each cell 352 has an inflow apex and an outflow apex that is not directly connected to any other structure of the frame 310. In between each adjacent pair of a sequential two cells 352 (e.g. near the circumferential center of each group of four cells 352), a support strut 354 extends to connect to an outflow apex of a cell 332 of the main body 330. With this configuration, the commissure ring 350 circumferentially supports the CAFs 340 to help maintain the CAFs 340 in a generally circular or cylindrical configuration. This functionality may help (i) prevent the CAFs 340 from deforming as the main body 330 section deforms and (ii) help buttress or prevent the CAFs 340 from canting or deflecting too far inwardly when the prosthetic leaflets close during ventricular systole, since the backpressure on the closed prosthetic leaflets will tend to pull the CAFs 340 radially inwardly. The use of supporting struts 354 at selected locations around the commissure ring 350, as opposed to having every cell 352 couple at its inflow end to a corresponding outflow end of a cell 332, may help to provide the desired support for CAFs 340 without too rigidly coupling the commissure ring to the main body 330. In other words, too rigid a coupling of the commissure ring 350 to the main body 330 might result in the commissure ring 350 getting deformed during beating of the heart and thus deforming the uniformity of the CAFs 340. By using only selected supporting struts 354, fine tuning between supporting the CAFs 340, without being too rigidly connected to the main body portion 330, may be achieved. Although commissure ring 350 is shown with a single row of cells 352, with four cells between each pair of CAFs 340, it should be understood that more rows of cells, and/or more or fewer cells between each pair of CAFs 340, may be used. The exact number, size, and/or shape of cells 352 may be adjusted to fine tune the desired amount of support of CAFs 340.

Although not shown in FIGS. 4-6, instead of forming the commissure ring 350 integrally with the frame 310, it may be formed separately and later attached to the frame 310 during assembly of the prosthetic heart valve 300. In this configuration, the commissure ring may be formed from a single row of diamond-shaped cells (or more rows, with the same, more, or fewer number of cells as shown with commissure ring 350), and may be positioned around the exterior of the CAFs 340 and later coupled to the CAFs 340 (e.g. via suturing through one or more holes in the CAFs 340. Additional designs of commissure rings that may be suitable for use in place of integrated commissure ring 350 (with or without additional modifications) are described in greater detail in U.S. Provisional Patent Application No. 63/384,521, filed Nov. 21, 2022 and titled “Transcatheter Prosthetic Atrioventricular Valve with Stiffening Structure.”

It is generally important for prosthetic heart valves to achieve sealing against the native valve annulus into which they are implanted. In other words, it is desirable that blood flow in the antegrade direction through the interior of the prosthetic heart valve when the prosthetic leaflets are open, and that no blood can flow through the prosthetic heart valve in the retrograde direction with the prosthetic leaflets are closed. If proper sealing is not achieved, blood may leak around the outside of the prosthetic heart valve, so that retrograde blood flow occurs even if the prosthetic leaflets are property coapted. As noted above, this may be referred to as PV leak, and the inclusion of a rotationally symmetric or uniform atrial flare 320 may increase the likelihood of PV leak compared to an asymmetric atrial flare that is specifically designed to match the contours of the native anatomy.

In order to help mitigate any potential PV leak, the prosthetic heart valve 300 may include one or more features. For example, a blood-impermeable cuff (e.g. fabric or tissue-based) may be positioned on the interior and/or exterior of the frame 310 at or around the atrial flare 320. In one example, a fabric cuff may be provided on the exterior of the atrial flare 320 to contact the native tissue and promote tissue ingrowth, which may help with long-term sealing. In another example, a tissue cuff may be provided on the interior surface of the atrial flare, to help ensure blood flowing into the prosthetic heart valve 300 is funneled to the interior of the prosthetic heart valve 300 and not around the exterior. In some embodiments, both cuffs may be provided.

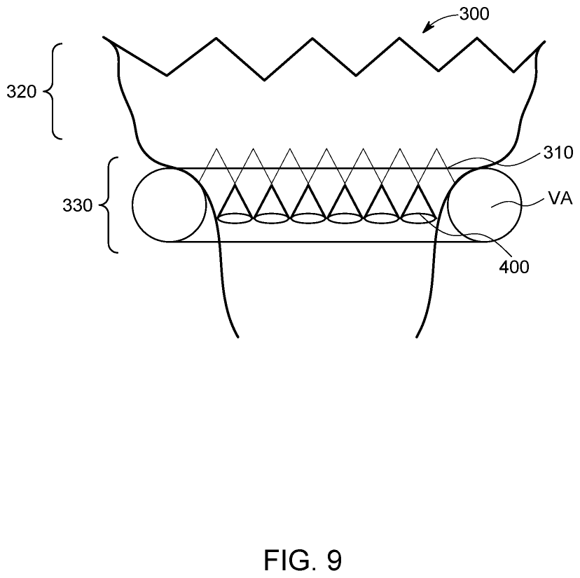

FIGS. 7-8 illustrate another feature that may be provided with prosthetic heart valve 300 to further mitigate PV leak, which may be provided instead of or in addition to any or all of the cuffs described above. FIGS. 7-8 illustrate an isolated portion of the frame 310, which be the portion intended to contact or be positioned adjacent to the native annulus. In the context of the specific frame 310 shown in FIGS. 4-6, the full diamond cells may generally correspond to cells 332 of the main body portion 330. The gap between adjacent pairs of cells 332 on the outflow side may be thought of as a “half-cell” that has the general shape of a half-diamond. In the illustrated embodiment, multiple blood impermeably fabric cups 400 are provided on the fame, with one cup 400 being positioned within each half-cell, although not all half-cells need to include a cup 400. The cups 400 may be formed in different specific ways, but generally each cup 400 includes a blood impermeable fabric that has at least two opposing sides that form an opening facing in the outflow direction and which are closed in the inflow direction. For example, referring to FIG. 8, in one particular configuration each cup 400 is formed of two generally triangular patches, with a luminal-facing patch 410 and an abluminal-facing patch 420. Each patch 410, 420 may be sutured to each the struts of the two adjacent cells 332 that form the half-cell in which the patches 410, 420 are positioned, but the outflow ends (toward the bottom in the view of FIG. 8) of each patch 410, 420 remain uncoupled from each other. In some embodiments, the patches 410, 420 are provided as separate pieces, and the two angled edges of patch 410 may be coupled (e.g. sutured) to the corresponding angled edges of patch 420, again while leaving the corresponding outflow edges unattached to each other. In other configurations, each cup 400 may be provided as a preformed member with patches 410, 420 forming part of one integral unit. In still other embodiments, the fabric (or other material, such as tissue) cups 400 may be provided not as individual units that must be individually attached to corresponding half-cells, but as a continuous unit that defines individual cups 400 along the continuous unit. Regardless of the particular way in which the cups 400 are manufactured, they work substantially the same in use. In particular, during ventricular diastole (represented in FIG. 7), the pressure in the left atrium is greater than the pressure in the left ventricle, causing blood to flow through the interior of the prosthetic heart valve 300 through the open prosthetic leaflets. However, during ventricular systole (represented in FIG. 8), the pressure in the left ventricle is greater than the pressure in the left atrium, causing the prosthetic leaflets of prosthetic heart valve 300 to close or coapt, while blood flows from the left ventricle, through the open aortic valve, and into the aorta. As this occurs, there is retrograde pressure R applied on the entire prosthetic heart valve 300 exposed to the left ventricle, and not just on the prosthetic leaflets. This retrograde pressure R will tend to cause blood to enter the cups 400 (as shown in FIG. 8) while moving the patches 410, 420 away from each other to “open” the cup 400. As these cups “open, the fabric-particularly the patch 420 facing in the abluminal direction, will tend to fill any gaps that may otherwise exist between the outer surface of the prosthetic heart valve 300 and the tissue of the native valve annulus confronting the outer surface of the prosthetic heart valve 300. FIG. 9 is a highly schematic illustration of the prosthetic heart valve 300 implanted into a patient's native valve annulus VA, showing the cups 400 generally aligned with the native valve annulus VA, and the retrograde pressure forcing the cups 400 to open and increase sealing with the interior of the native valve annulus VA. Although one complete row of cups 400 are shown, it should be understood that a single row of cups 400 may be provided with that row not being a complete row, and more rows of cups (complete and/or incomplete rows) may be provided as desired. Examples of cups similar to cups 400 are described in more detail in U.S. Pat. No. 9,913,715, the disclosure of which is hereby incorporated by reference herein.

Although fabric cups 400 are one feature (e.g. in addition to other interior and/or exterior cuffs on the frame 310 of the prosthetic heart valve) that may assist with mitigating PV leak, still other options may be suitable. For example, a loose fabric, which is formed to be impermeable to blood flowing through the fabric, may be coupled (e.g. sutured) to the outside perimeter of the frame 310. That loose fabric may extend into the left ventricle, and that fabric may fill and expand during ventricular diastole due to the pressure differential between the left ventricle and the left atrium. When filled, not unlike cups 400, the loose fabric would expand to fill any gaps created between the frame and native anatomy. Preferably, the length of the fabric is chosen to minimize the likelihood that the fabric could block the LVOT.

Although one particular example of prosthetic heart valve 300 is described above, it should be understood that modifications to prosthetic heart valve 300 may be made. For example, prosthetic heart valve 300 could be used with a valve assembly that includes two prosthetic leaflets, instead of three. In such a configuration, the frame 310 may be modified, for example to include two CAFs 340. And while prosthetic heart valve 300 may include prosthetic leaflets such as prosthetic leaflets 138, other prosthetic leaflets (including synthetic fabric leaflets) may be used with prosthetic heart valve 300. Further, although frame 310 is shown as having diamond-shaped cells in a particular number of rows and number of cells per row, other shape cells, and other numbers of rows and numbers of cells per row, may be suitable, as long as the frame 310 retains the ability to collapse and expand.

In use, prosthetic heart valve 300 may be delivered using a known delivery device, for example including a delivery device and/or system such as that described in U.S. Pat. Nos. 9,526,611 or 10,667,905, the disclosures of which are hereby incorporated by reference herein. However, the delivery devices in the two patents referenced above are configured for use with a dual-framed prosthetic mitral valve. Because the single-framed prosthetic mitral valve 300 described herein (as well as variants thereof) is less bulky than dual-framed prosthetic heart valves, the prosthetic mitral valve 300 may be suited for delivery with a reduced sheath outer diameter that could interface with prosthetic heart valve 300 since this design reduces the amount of material required to compact the prosthetic heart valve 300 into the loading tube/delivery sheath compared to prior delivery devices. For example, prosthetic heart valve 300 may be delivered using a delivery device that has catheter sized between about 28 French (9.33 mm) to about 30 French (10 mm) outer diameter.

Finally, it should be understood that prosthetic heart valve 300 is designed for use as part of a system that includes a tether (such as tether 226 or other known prosthetic mitral valve tethers) and an anchor (including anchor 210 or other known prosthetic mitral valve epicardial anchors).

Although particular designs for prosthetic heart valve 300 are provided above, other designs may be suitable. For example, instead of three axially-extending struts 360, the bottom or outflow end of the frame 310 may have a design more similar to that of inner frame 140, for example three or more (including four, five, or six) struts that converge to a tether connecting portion 144 that is formed by the ends of the struts, with adjacent struts connected by one or more “V”-shaped connectors to form a continuous cylindrical section for receiving the tether (as opposed to struts that are not directly connected to each other in the circumferential direction).

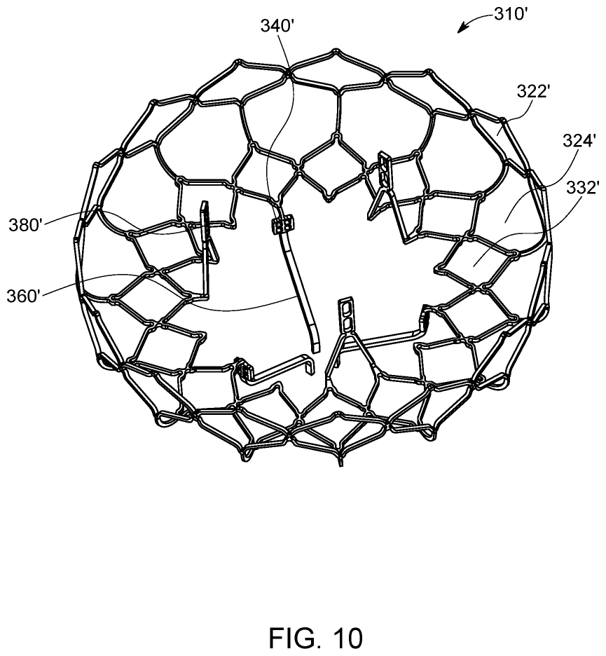

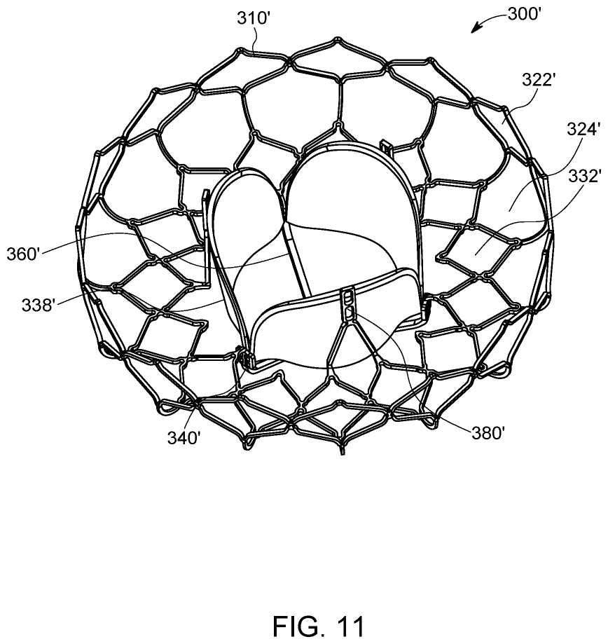

FIGS. 10-11 show a prosthetic heart valve 300′ (or components thereof) that has some features in common with prosthetic heart valve 300. For example, prosthetic heart valve 300′ may include a single, self-expanding frame 310′ that may be generally similar to frame 310, with the exception of certain geometrical considerations. For example, frame 310′ may include a top or inflow-most circumferential row of cells 322′, which may be generally diamond- or hexagon-shaped. Frame 310′ may include a second or transition circumferential row of cells 324′, which may be generally diamond- or hexagon-shaped, and which may define a large area compared to cells 322′ and 332′ (described below). In the expanded condition (e.g. in the absence of applied forces), the transition cells 324′ may taper radially inwardly from the inflow-most row of cells 322′ toward the outflow-most row of cells 332′. Frame 310′ may include an outflow-most circumferential row of cells 322′, which may be generally diamond- or hexagonal-shaped.

Similar to frame 310, frame 310′ includes three CAFs 340′ (at least in an embodiment with three prosthetic leaflets 338′). In the illustrated embodiment, each CAF 340′ includes a substantially rectangular body with one or more eyelets for receiving sutures. In the illustrated embodiment, each CAF 340′ includes a two-by-two arrangement of eyelets. Each CAF 340′ may be positioned at an outflow end (e.g. via a linking strut) of a cell 332′ in the outflow-most row of cells. A tether strut 360′ may extend in an outflow direction from each CAF 340′, similar to the relationship between struts 360 and CAFs 340. In the illustrated embodiment, each tether strut 360′ extends radially inwardly and in the outflow direction in the expanded condition of the frame 310′, so that an end of a tether may be positioned within, and coupled to, the tether struts 360′. Although not shown, the tether struts 360′ may include apertures similar to those provided with tether struts 360.

One of the major distinctions between prosthetic heart valve 300′ and 300 is that frame 310′ includes a plurality of posts 380′ for supporting bellies of the prosthetic leaflets 338′. In the illustrated embodiment, three posts 380′ are provided (corresponding to three prosthetic leaflets 338′). Each post 380′ may be positioned extending from a terminal end of a diamond-shaped cell that includes four struts, two of which are shared with a cell 332′ in the outflow-most row, and two of which extend back in the inflow direction in the expanded condition of the frame 310′, with the post 380′ also extending in the inflow direction. In the illustrated embodiment, each post 380′ includes two apertures in a single column, although more or fewer apertures may be provided, in similar or different configurations. In the illustrated embodiment, frame 310′ includes three CAFs 340′ at substantially equal intervals around the frame 310′ and three posts 380′ at substantially equal intervals around the frame 310′, with the posts 380′ offset from the CAFs 340′ by about 60 degrees.

As best shown in FIG. 11, the posts 380′ may be configured to provide support to the bellies of the prosthetic leaflets 338′ (the leaflet bellies generally being the contoured portions of the leaflets opposite the free edges that coapt with each other to create the valve functionality). In the embodiment of FIG. 11, since each post 380′ is staggered between a pair of adjacent CAFs 340′, each post 380′ may generally align with the peak of the belly of a corresponding prosthetic leaflet 338′. In some embodiments, the prosthetic leaflets 338′ may be directly couped to the posts 380′ (e.g. via suturing). In other embodiments, a tissue or fabric cylinder may be positioned within the posts 380′ and directly coupled (e.g. via suturing) to the posts 380′, with the bellies of the prosthetic leaflets 380′ being directly coupled (e.g. via suturing) to the intermediate tissue or fabric cylinder. In still other embodiments, a separate ring frame, such as the non-integral version of commissure ring 350 described above, may be positioned to circumscribe the outer surfaces of the posts 380′, with the ring providing additional stability to the posts 380′, which in turn may be directly coupled to the prosthetic leaflets 338′. It should be understood that this ring, although having the same or similar structure as a “commissure” ring described above, would function as a “leaflet belly” ring. Although any of these options may be suitable, the motion and loading at the leaflet belly is typically smaller than that nearer the commissures, so a tissue or fabric tube may provide more than enough support, although the other options (e.g. leaflet belly ring) are still viable options.

One of the benefits of prosthetic heart valve 300′ is that there is only minimal structure that extends into the left ventricle, which is generally desirable in order to reduce the likelihood of obstructing the LVOT. For example, instead of a commissure ring 350 being positioned in the left ventricle, the posts 380′ (and any corresponding fabric or tissue tube or leaflet belly ring) is positioned within the native valve annulus (and/or on the atrial side thereof). Thus, by having a single frame 310′, the prosthetic heart valve 300′ may be smaller than double-framed embodiments and able to treat patients with smaller mitral anatomy. At the same time, the presence of structure of the prosthetic heart valve 300′ is minimized, while support is provided to the prosthetic leaflets 338′ to help them maintain the desired opening and closing shapes as the heart cycles between systole and diastole, despite the significant pressure applied to the prosthetic leaflets 338′ and the frame 310′ during normal operation of the heart.

As with prosthetic heart valve 300, it should be understood that additional “soft” components, such as inner cuffs, outer cuffs, and/or PV leak mitigation features such as those shown and described in connection with FIGS. 7-9, may also be applied to prosthetic heart valve 300′.

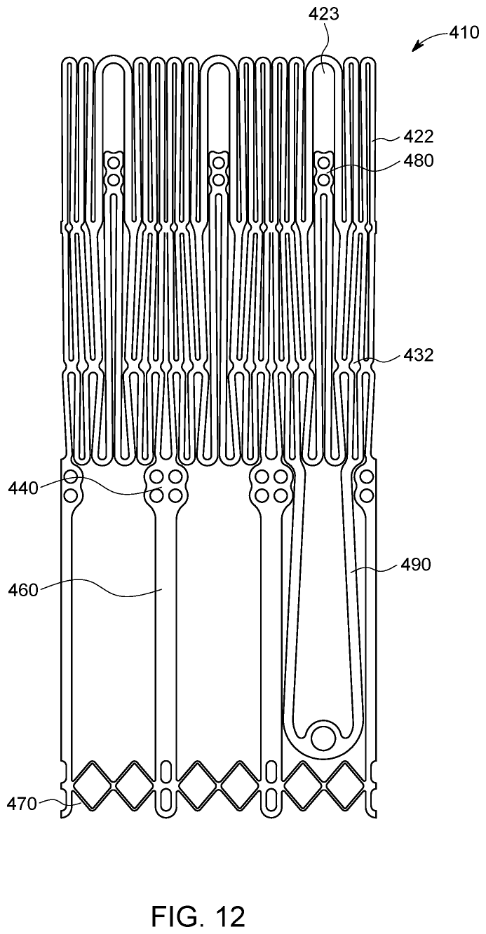

FIG. 12 illustrates a cut pattern for a frame 410 that may be used in place of frame 310′ for prosthetic heart valve 300′. It should be understood that frame 410 is illustrated as if cut longitudinally and laid out on a table in an unexpanded state. Frame 410 may include an outflow-most row of cells 422 that may be generally diamond-shaped when in the expanded state, and which may generally flare radially outwardly as with other frames described herein. Frame 410 may include an adjacent row of cells 432 that may be generally diamond-shaped when in the expanded condition, and which, when expanded, may taper to a smaller diameter compared to the atrial or inflow-most section of frame 410. Similar to frame 310′, frame 410 includes three CAFs 440 (and tether struts 460 extending from the CAFs 440) that are offset about 60 degrees from posts 480. Posts 480 may have a similar shape and function as posts 380′. However, posts 480 may be coupled to two struts that nest within, and are coupled to, an enlarged cell 423 when the frame 410 is collapsed. Frame 410 may include three enlarged cells 423 (e.g., if three prosthetic leaflets and three corresponding posts 480 are provided). The enlarged cells 423 may extend the length of both cells 422, 432. In the deployed or expanded condition, the posts 480 (and their two connecting struts) point toward the inflow end of the frame 410 and extend radially inwardly, in substantially similar fashion as posts 380′ shown in FIGS. 10-11. As with prosthetic heart valve 300′, a prosthetic heart valve incorporating frame 410 may include a fabric or tissue support tube coupled to the interior of the posts 480, or a leaflet-belly ring coupled to the exterior of the posts 480, to help support the prosthetic leaflet bellies. In some embodiments, as described with posts 380′, the posts 480′ may be directly coupled to the prosthetic leaflet bellies.

In addition to the specific configuration of posts 480, frame 410 has two additional major differences compared to frame 310′. First, the outflow ends of the tether struts 460 are circumferentially connected to each other via cells 470, which may be generally diamond shaped. Cells 470 are conceptually similar to the V-shaped struts at the connection portion 144 of inner frame 140, but are formed as diamond cells 470. With this configuration, the outflow end of the frame 410 forms a generally continuous cylinder through which an end of the tether may be placed, and then the outflow end of the frame may be compressed over the tether and coupled to the tether (including via sutures, adhesives, etc.), to help better secure the tether to the frame 410. It should be understood that features like cells 470 may be applied to other embodiments of frames with tether struts described herein. The second additional main difference is that frame 410 may include a leaflet clip 490, although it should be understood that leaflet clip 490 may be omitted form frame 410 (and similarly, other frames described herein may include a leaflet clip like leaflet clip 490). Leaflet clip 490 may be generally in the form of an enlarged partial cell formed by two struts extending from the outflow end of cells 432, with the partial cell extending nearly the entire distance between the row of cells 432 and the cells 470, and occupying most of the area between two adjacent tether struts 460, when the frame 410 is collapsed. In some embodiments, only a single leaflet clip 490 is provided, making the frame 410 asymmetric, at least at the outflow end. Clip 490 may be shape set so that, when frame 410 is expanded, the leaflet clip 490 extends radially outwardly and hooks up toward the inflow end of the frame 410. With this configuration, the prosthetic heart valve that incorporates frame 410 may be implanted in a desired rotational orientation so that the leaflet clip 490 aligns with the native anterior mitral valve leaflet (and preferably the A2 segment thereof), and acts to clip or otherwise immobilize the native anterior mitral valve leaflet between the leaflet clip 490 and portions of the exterior of frame 410. By immobilizing the native anterior mitral valve leaflet, the risk of that anterior leaflet obstructing the LVOT may be reduced. As with frame 310′, frame 410 may reduce the amount of structure within the LVOT generally, and if the optional leaflet clip 490 is provided, the likelihood of LVOT obstruction may be reduced even further.

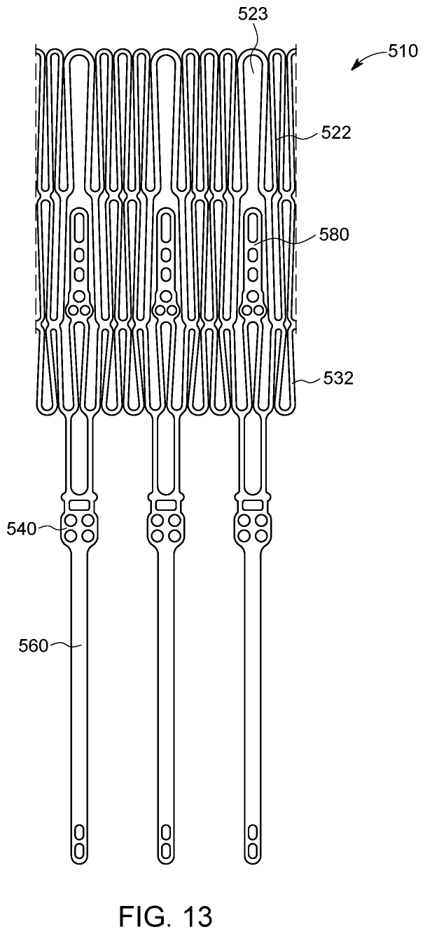

FIG. 13 illustrates a cut pattern for a frame 510 that may be used in place of frame 310′ or 410 for prosthetic heart valve 300′ or a similar prosthetic heart valve. It should be understood that frame 510 is illustrated as if cut longitudinally and laid out on a table in an unexpanded state. Frame 510 may include an outflow-most row of cells 522 that may be generally diamond-shaped when in the expanded state, and which may generally flare radially outwardly as with other frames described herein. Frame 510 may include an adjacent row of cells 532 that may be generally diamond-shaped when in the expanded condition, and which, when expanded, may taper to a smaller diameter compared to the atrial or inflow-most section of frame 510. Similar to frames 310′ and 410, frame 510 includes three CAFs 540 (and tether struts 560 extending from the CAFs 540). However, CAFs 540 and posts 580 are axially aligned, instead of being offset as in frames 310′ and 410. Posts 580 may have a generally similar shape and function as posts 380′ and 480. Similar to posts 480, posts 580 may be coupled to two struts that nest within, and are coupled to, an enlarged cell 523 when the frame 510 is collapsed. Frame 510 may include three enlarged cells 523 (e.g., if three prosthetic leaflets and three corresponding posts 580 are provided). The enlarged cells 523 may extend about half the combined length of cells 522 and 532, although other lengths may be suitable. In the deployed or expanded condition, the posts 580 (and their two connecting struts) point toward the inflow end of the frame 510 and extend radially inwardly, in substantially similar fashion as posts 380′ and 480 shown in FIGS. 10-12. As with prosthetic heart valve 300′, a prosthetic heart valve incorporating frame 510 may include a fabric or tissue support tube coupled to the interior of the posts 580, or a leaflet-belly ring coupled to the exterior of the posts 580, to help support the prosthetic leaflet bellies. Because the posts 580 are axially aligned with the CAFs 540, the posts 580 will not align with the peak of the prosthetic leaflet bellies, and thus it may not be desirable to forego the fabric or tissue tube (or the leaflet belly ring). Although frame 510 is shown without a leaflet clip and without diamond-shaped cells at the outflow end of the tether struts 560, either or both may be included with frame 510. Otherwise, besides minor differences in the shape of posts 580, CAFs 540, and the extended struts connecting CAFs 540 to cells 532, frame 510 may be generally similar in structure and function as frame 410.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic atrioventricular valve system comprising:
a prosthetic atrioventricular valve including:
a self-expandable frame having a collapsed condition and an expanded condition, wherein in the expanded condition the frame includes (i) a body portion sized to be positioned within, and in contact with, a native atrioventricular valve annulus, (ii) an atrial flare portion positioned in an inflow direction relative to the body portion, the atrial flare portion having an outer diameter that is greater than an outer diameter for the body portion, (iii) a plurality of commissure attachment features extending in an outflow direction from the body portion, and (iv) a commissure support ring coupled to and circumferentially supporting the plurality of commissure attachment features; and
a plurality of prosthetic leaflets mounted to the plurality of commissure attachment features,
wherein the body portion, the atrial flare portion, and the plurality of commissure attachment features are integral with each other.

2. The prosthetic atrioventricular heart valve system of claim 1, wherein the commissure support ring is integral with the body portion.

3. The prosthetic atrioventricular heart valve system of claim 2, wherein the commissure support ring is formed by at least one row of diamond-shaped cells.

4. The prosthetic atrioventricular heart valve system of claim 3, wherein the cells of the commissure support ring are positioned between adjacent pairs of the plurality of commissure attachment features.

5. The prosthetic atrioventricular heart valve system of claim 4, wherein the at least one row of diamond-shaped cells forming the commissure support ring is the only row of diamond-shaped cells forming the commissure support ring.

6. The prosthetic atrioventricular heart valve system of claim 4, wherein each cell of the commissure support ring has an inflow apex, and no inflow apex of any cell of the commissure support ring is directly coupled to the body portion.

7. The prosthetic atrioventricular heart valve system of claim 6, wherein selected struts directly couple the commissure support ring to the body portion, the selected struts attaching to the commissure support ring at locations between selected circumferentially adjacent pairs of cells of the commissure support ring.

8. The prosthetic atrioventricular heart valve system of claim 2, wherein the frame is a first frame, and the prosthetic atrioventricular valve does not include a second frame.

9. The prosthetic atrioventricular heart valve system of claim 2, wherein the commissure support ring is not integral with the frame.

10. The prosthetic atrioventricular heart valve system of claim 9, wherein the commissure support ring is assembled to the frame so that the commissure support ring is aligned with, and radially outside of, the plurality of commissure attachment features.

11. The prosthetic atrioventricular heart valve system of claim 1, wherein the frame includes a plurality of tether struts, and the system includes a tether secured the plurality of tether struts at a terminal end of the plurality of tether struts.

12. The prosthetic atrioventricular heart valve system of claim 11, wherein the plurality of tether struts are integral with the frame, each of the plurality of tether struts extending in the outflow direction from a corresponding one the plurality of commissure attachment features.

13. The prosthetic atrioventricular heart valve system of claim 12, wherein the plurality of tether struts includes exactly three tether struts and the plurality of commissure attachment features includes exactly three commissure attachment features.

14. The prosthetic atrioventricular heart valve system of claim 11, wherein the plurality of tether struts are integral with the commissure support ring, each of the plurality of tether struts extending in the outflow direction form the commissure support ring.

15. The prosthetic atrioventricular heart valve system of claim 11, wherein each of the tether struts terminates at a free end, each of the free ends including at least one aperture for receiving a connecting suture therethrough.

16. The prosthetic atrioventricular heart valve system of claim 11, wherein the system includes an epicardial anchor configured to attach to the tether.

17. The prosthetic atrioventricular heart valve system of claim 1, further comprising a skirt coupled to an exterior surface of the atrial flare portion.

18. The prosthetic atrioventricular heart valve system of claim 1, wherein the atrial flare portion is rotationally symmetric.

19. The prosthetic atrioventricular heart valve system of claim 1, further comprising a plurality of cups coupled to the body portion, each of the plurality of cups being coupled to a cell of the bod portion and including two patch portions, the two patch portions begin coupled to each outer at an inflow portion of the two patch portions, but uncoupled to each other at an outflow end portion, so that retrograde blood may flow into each cup via the uncoupled outflow end portion to force the two patch portions to billow away from each other.

20. The prosthetic atrioventricular heart valve system of claim 19, wherein the two patch portions of each of the plurality of cups are generally triangular.
